(19) United States
(12) Patent Application Publication
(10) Pub. No.: US 2005/0069502 A1
(43) Pub. Date: Mar. 31, 2005

(54) Hydrophobic polymer carrier based liquid tooth whitening composition

(76) Inventors: Suman K. Chopra, Dayton, NJ (US); Lynette A. Zaidel, Cranford, NJ (US); Guisheng Pan, Spotswood, NJ (US); Suryakant Patel, Flemington, NJ (US); Ryan B. Cameron, Somerset, NJ (US); Michael Prencipe, West Windsor, NJ (US)

Correspondence Address: HARNESS, DICKEY, & PIERCE, P.L.C, 7700 BONHOMME, STE 400, ST. LOUIS, MO, 63105, US

(21) Appl. No.: 10/917,263

(22) Filed: Aug. 12, 2004

Related U.S. Application Data
(63) Continuation-in-part of application No. 10/642,458, filed on Aug. 15, 2003.

(51) Int. Cl. A61K 7/20

(52) U.S. Cl. 424/53

(57) ABSTRACT

A tooth whitening system comprising a tooth whitening composition comprising a whitening agent and a hydrophobic polymer carrier. The tooth whitening composition is preferably substantially non-water soluble. The tooth whitening composition is applied to the surface of a tooth to be whitened in a mammal to effect whitening on the tooth surface. Methods of whitening a surface of a tooth are also provided.

### CROSS-REFERENCE To RELATED APPLICATIONS

This application is a continuation-in-part of U.S. patent application Ser. No. 10/642,458 filed on Aug. 15, 2003. The disclosure of the above application is incorporated herein by reference.

### INTRODUCTION

The present invention relates to tooth whitening systems employing compositions comprising hydrophobic polymers. Embodiments of the present invention include tooth whitening compositions comprising a hydrophobic polymer carrier and a whitening agent.

It has become desirable for a person's teeth to appear bright or “white”. Society places a high value on the “whiteness” of one's teeth. One whose teeth are white may enjoy more personal confidence and satisfaction and may even enjoy greater social acceptance.

In a mammal, a tooth is comprised of an inner dentin layer and an outer hard enamel layer that is the protective layer of the tooth. The enamel layer of a tooth is naturally an opaque white or slightly off-white color. It is the enamel layer that can become stained or discolored. The enamel layer of a tooth is composed of hydroxyapatite mineral crystals that create a somewhat porous surface. These hydroxyapatite crystals form microscopic hexagonal rods or prisms that make up the enamel surface. As a result, the surface of the enamel layer presents microscopic spaces or pores between the prisms. It is believed that this porous nature of the enamel layer is what allows staining agents and discoloring substances to permeate the enamel and discolor the tooth. These remaining substances can occupy the microscopic spaces and eventually alter the color of the tooth.

Many substances that a person confronts or comes in contact with on a daily basis can “stain” or reduce the “whiteness” of one's teeth. In particular, the foods, tobacco products and fluids that one consumes tend to stain one's teeth. These products or substances tend to accumulate on the enamel layer of the tooth and form a pellicle film over the teeth. These staining and discoloring substances can then permeate the enamel layer. This problem occurs gradually over many years, but imparts a noticeable discoloration of the enamel of one's teeth. So long as the discolored teeth are still healthy and do not pose any health risk or problem, a product or substance that would whiten the discolored teeth would be advantageous.

It is also essential that a tooth whitening product that is to be used at home or in private by the consumer be safe and easy to use and be stable and retain its whitening efficacy during its storage on retail store shelves as well as over the period of use by the consumer.

Products and substances that are presently available to whiten teeth include a variety of different ingredients, but the primary active ingredient is a peroxide agent formulated into an aqueous liquid, solution, paste or gel carrier. These products potentially lose their whitening efficacy over time as peroxide compounds in aqueous solutions are relatively unstable. This tendency toward instability of peroxide during storage has limited the utility of aqueous liquid whitening products for whitening teeth. It would be highly desirable, therefore, to provide a stable peroxide whitening liquid to effect substantive whitening.

### SUMMARY

The present invention provides tooth whitening systems for whitening a surface of a tooth. Embodiments include a tooth whitening composition comprising a whitening agent and a hydrophobic polymer carrier, wherein the whitening composition adheres to the tooth surface to effect whitening thereof.

In another embodiment, the present invention provides a method of whitening a surface of a tooth in a mammal, comprising applying to the tooth a whitening composition comprising a whitening agent and a hydrophobic polymer carrier.

It has been discovered that compositions and methods of this invention afford advantages over whitening compositions among those known in the art including one or more of: enhanced whitening efficacy, providing a higher available concentration of whitening agent, stability of the whitening agent during storage, adherence of the whitening composition to the tooth surface even in the presence of saliva and sustained and controlled delivery of the whitening agent for a longer duration of time Further uses, benefits and embodiments of the present invention are apparent from the description set forth herein.

### DESCRIPTION

The following definitions and non-limiting guidelines must be considered in reviewing the description of this invention set forth herein. The headings (such as “Introduction” and “Summary,”) and sub-headings (such as “Whitening Compositions” and “Methods”, for example) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being an “active” or a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features. Specific Examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word ‘include,” and its variants, is intended to be non-limiting, such that recitation of items in a list is not to the exclusion of other like items that may also be useful in the materials, compositions, devices, and methods of this invention.

As referred to herein, all compositional percentages are by weight of the total composition, unless otherwise specified.

As referred to herein, “tooth” or “teeth” refers to natural mammalian teeth, dentures, dental plates, fillings, caps, crowns, bridges, dental implants, and the like, and any other hard surfaced dental prosthesis either permanently or temporarily fixed within the oral cavity. As used herein, “whitening” refers to a change in visual appearance of a tooth, preferably such that the tooth has a brighter shade. Increase in whiteness of a dental surface can be observed visually, for example with the aid of color comparison charts or gauges, or measured by colorimetry, using any suitable instrument such as a Minolta Chromameter, e.g., model CR-400 (Minolta Corp., Ramsey, N.J.). The instrument can be programmed, for example, to measure Hunter Lab values or L*a*b* values according to the standard established by the International Committee of Illumination (CIE). The L*a*b* system provides a numerical representation of three-dimensional color space where L* represents a lightness axis, a* represents a red-green axis and b* represents a yellow-blue axis. The L* and b* axes are typically of greatest applicability to measurement of tooth whiteness. Increase in whiteness can be computed from differences in L*, a* and b* values before and after treatment, or between untreated and treated surfaces. A useful parameter is ΔE*, calculated as the square root of the sum of the squares of differences in L*, a* and b* values, using the formula:
ΔE*=[(ΔL*)²+(Δa*)²+(Δb*)²]^{1/2}

A higher value of ΔE* indicates greater increase in whiteness. In various embodiments, the method of the present invention can effect a ΔE* of at least about 1, or at least about 3, or at least about 4, or at least about 5.

### Tooth Whitening System

In various embodiments, the present invention provides an improved tooth whitening system that employs a tooth whitening composition that adheres to a surface of a tooth to be whitened. The tooth whitening composition comprises a whitening agent and a hydrophobic polymer carrier. In various embodiments, the hydrophobic polymer carrier of the whitening composition is preferably anhydrous and provides a stable vehicle or carrier for the whitening agent during storage and before use. The whitening composition will gradually dissolve or disintegrate upon exposure to the saliva in the aqueous oral cavity environment, thus providing delivery of the whitening agent to a tooth surface. In some embodiments, it is preferred that the hydrophobic polymer carrier is non-water soluble and thus is stable for longer durations during exposure to saliva and other aqueous solutions found in an oral cavity, as compared to prior art water-soluble whitening solutions.

In various embodiments, the whitening composition can be incorporated into a dental strip or film form, or into a liquid product which can be applied to the surface of a tooth by manual application. Upon application to the surface of a tooth, the applied whitening composition forms an adherent layer of whitening agent containing product that has the capacity to release the whitening agent over an extended period of time, e.g., from about 5 to about 45 minutes. The applied layer adheres to the tooth surface and the released whitening agent effects whitening of the tooth surface to which the composition is applied.

In another embodiment, methods of whitening a surface of a tooth are provided.

### Whitening Compositions

The present invention provides a whitening composition for use in a tooth whitening system. In one embodiment, the whitening composition comprises a whitening agent and a hydrophobic polymer carrier. The whitening composition may further comprise an adhesion enhancing agent. Other additional ingredients that may be added include those known to one of skill in the art, including one or more of the following components: surfactants, flavoring agents, sweeteners, desensitizing agents, antimicrobial agents, anti-caries agents, anti-calculus agents, anti-inflammatory agents, vitamins, pigments and coloring agents, and enzymes, as will be discussed in greater detail below.

Active ingredients useful herein are optionally present in the compositions of the present invention in safe and effective amounts. A “safe and effective” amount of an active is an amount that is sufficient to have the desired therapeutic or prophylactic effect in the human or lower animal subject to whom the active is administered, without undue adverse side effects (such as toxicity, irritation, or allergic response), commensurate with a reasonable benefit/risk ratio when used in the manner of this invention. The specific safe and effective amount of the active will vary with such factors as the particular condition being treated, the physical condition of the subject, the nature of concurrent therapy (if any), the specific active used, the specific dosage form, the carrier employed, and the desired dosage regimen.

### Whitening Agents

In various embodiments, the compositions of the present invention comprise a whitening agent as the main active ingredient. As further discussed below, a “whitening agent” is a material which effects whitening of a tooth surface to which it is applied. In various embodiments, the whitening compositions of this invention comprise a whitening agent selected from the group consisting of peroxides, chlorites, and hypochlorites. Examples of suitable chlorites and hypochlorites include those having alkali or alkaline metal cations and include calcium chlorite, barium chlorite, magnesium chlorite, lithium chlorite, sodium chlorite, potassium chlorite, calcium hypochlorite, barium hypochlorite, magnesium hypochlorite, lithium hypochlorite, lithium hypochlorite, and sodium hypochlorite.

In one embodiment, the whitening agent comprises a peroxide compound. As referred to herein, a “peroxide compound” is an oxidizing compound comprising a bivalent oxygen-oxygen group. Peroxide compounds include peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound comprises hydrogen peroxide, urea peroxide, sodium percarbonate and mixtures thereof.

Peroxide releasing compounds particularly useful in the whitening compositions of the present invention include peroxide containing compounds such as urea peroxide, sodium percarbonate, sodium perborate and polyvinylpyrrolidone-H₂O₂ complexes (hereinafter “PVP-H₂O₂”). Polyvinylpyrrolidone is also known as poly-N-vinyl-poly-2-pyrrolidone and commonly abbreviated to “PVP”. PVP generally refers to a polymer containing vinylpyrrolidone (also referred to as N-vinylpyrrolidone, N-vinyl-2-pyrrolidione and N-vinyl-2-pyrrolidinone) as a monomeric unit. The monomeric unit consists of a polar imide group, four non-polar methylene groups and a non-polar methane group.

Both linear and cross-linked complexes of PVP-H₂O₂ are known in the art and are disclosed in U.S. Pat. No. 3,376,110 and U.S. Pat. No. 3,480,557, which are herein incorporated by reference, and have been used in compositions for treating acne vulgaris (U.S. Pat. No. 5,122,370). PVP-H₂O₂ complexes are also disclosed in U.S. Pat. No. 5,122,370. PVP-H₂O₂ is stable in an anhydrous environment. Upon exposure to highly aqueous environments, such as in the oral cavity, the PVP-H₂O₂ dissociates into individual species (PVP polymer and H₂O₂). In one embodiment, the PVP-H₂O₂ complex is about 80% by weight polyvinylpyrrolidone and 20% by weight H₂O₂.

In alternate embodiments, the whitening agent comprises a liquid peroxide solution. The hydrophobic polymer carrier of the whitening composition provides sufficient stability to permit the use of a liquid hydrogen peroxide. The liquid hydrogen peroxide comprises H₂O₂ generally contained in an aqueous water-based solution. In some embodiments, the liquid hydrogen peroxide has a concentration of peroxide to the total solution of about 0.5 to about 10%, more preferably 0.5 to 5% by weight). Additionally, a stabilizer may be present. For example, a 3% hydrogen peroxide solution with about 0.1 to about 0.5% of a stabilizer may be used. Acetanilide or a similar organic material can also be used with a pyrophosphate stabilizer such as sodium acid pyrophosphate (about 0.1 to about 1.0%) with a preferred amount of about 0.5%.

In certain embodiments, an agent to enhance release of the peroxide in the oral cavity is present as a part of the peroxide component whitening agent. Polypore® which is an allyl methacrylate crosspolymer available from Amcol health & Beauty Solutions, Inc. is such an enhancing agent.

In various embodiments, the whitening agent of the whitening composition comprises from about 0.1% to about 50%, optionally from about 0.5% to about 50%, and optionally from about 10% to about 40% of the oral care composition.

### Hydrophobic Polymer Carrier

The present invention preferably comprises a carrier that comprises a hydrophobic polymer. The term “hydrophobic” or “water-insoluble” as applied to polymers and as employed herein refers to an organic polymer which is substantially non-aqueous having a water solubility of less than one gram per 100 grams of water at 25° C. In various embodiments, a hydrophobic polymer is compatible with the whitening agents previously described above. In certain embodiments, a hydrophobic polymer is selected for the carrier to produce a tooth whitening composition having a viscosity of greater than about 1,000 centipoise (cP) and less than about 900,000 cP, preferably greater than about 10,000 cP and less than about 100,000 cP.

One preferred class of hydrophobic polymers comprise siloxane polymers, which are also generally known in the art as “silicone” polymers. In certain embodiments of the present invention, the hydrophobic polymers in the carrier are those in which a whitening agent can be dispersed and are well known in the art. Many such silicone polymers are commercially available. In various embodiments, a preferred silicone-based hydrophobic polymer is a polyorganosiloxane. One such polyorganosiloxane is produced by condensing a silicone resin and an organosiloxane such as a polydiorganosiloxane. Such hydrophobic polymers are an elastomeric, tacky material, adhesion of which to dental enamel surfaces can be varied by altering the ratio of silicone resin to polydiorganosiloxane in the copolymer molecule. Preferably, the polymers are pressure sensitive hydrophobic polymers specifically designed for pharmaceutical use and are permeable to many drug compounds and find application for the transdermal application of various compounds. In one such embodiment, the silicone polymers are the copolymer product of mixing a silanol terminated polydiorganosiloxane such as polydimethyl siloxane with a silanol-containing silicone resin whereby the silanol groups of the polydiorganosiloxane undergo a condensation reaction with the silanol groups of the silicone resin so that the polydiorganosiloxane is lightly crosslinked by the silicone resin (that is, the polydiorganosiloxane chains are bonded together through the resin molecules to give chain branching and entanglement and/or a small amount of network character) to form the silicone hydrophobic polymers. A catalyst, for example, an alkaline material, such as ammonia, ammonium hydroxide or ammonium carbonate, can be mixed with the silanol-terminated polydiorganosiloxane and the silicone resin to promote this crosslinking reaction. By copolymerizing the silicone resin with the silanol terminated polydiorganosiloxane, there results a polymer with self adhering properties and the cohesive properties of a soft elastomer matrix characteristic of pressure sensitive polymers being distinguished from the hard, non-elastomeric properties of other silicone resins. In one embodiment, hydrophobic polymers used in the carrier are available from the Dow-Corning Company under the brand name BIO-PSA.

The modification of a ratio of silicone resin to polydiorganosiloxane modifies the tackiness of the hydrophilic polymer. This ratio can be in the range of about 70:30 to about 50:50. For example, the BIO-PSA silicone sold by Dow-Corning is available in three silicone resin to silicone polymer ratios namely, 65/35 (low tack), 60/40 (medium tack), 55/45 (high tack). Such a polyorganosiloxane pressure sensitive adhesive is available dissolved in either ethyl acetate solvent or dimethicone.

In various embodiments, the hydrophobic polymer carrier is preferably present in the liquid whitening compositions of the present invention at a concentration of from about 1 to about 80% (by weight), optionally from about 15% to about 80%, optionally from about 15% to about 80%. In one embodiment, the hydrophobic polymer carrier is present at a level of from about 15% to about 40%. In one embodiment, the hydrophobic carrier is present at a level of from about 40% to about 80 %.

### Adhesion Enhancing Agents

In one embodiment of the present invention, the whitening composition further comprises an adhesion enhancing agent, that augments adhesion of the anhydrous whitening composition to the surface of the tooth, i.e., adhesion to the enamel. Adhesion enhancing agents useful with the present invention include inorganic materials as well as organic natural and synthetic polymers. Inorganic materials include amorphous silica compounds which function as thickening agents, and include colloidal silica compounds available under trademarks such as Cab-o-sil fumed silica manufactured by Cabot Corporation and distributed by Lenape Chemical, bound Brook, N.J.; Zeodent 165 from J. M. Huber Chemicals Division, Havre de Grace, Md. 21078; and Sylox 15 also known as Sylodent 15, available from Davison Chemical Division of W. R. Grace Corporation, Baltimore, Md. 21203. In certain embodiments, the inorganic adhesion enhancing material, such as silica, is surface treated to compatibilize the adhesion enhancing agent with the hydrophobic components in the whitening composition.

Organic materials which may be included in the compositions of the present invention to enhance the properties of the hydrophobic polymers of the present invention include adhesion enhancing agents such as waxes, inclusive of bees' wax, mineral oil, plastigel, (a blend of mineral oil and polyethylene), petrolatum, white petrolatum, shellac, versagel (blend of liquid paraffin, butene/ethylene/styrene hydrogenated copolymer) polyethylene waxes, microcrystalline waxes, polyisobutene, polyvinyl pyrrolidone/vinyl acetate copolymers, and insoluble polyacrylate copolymers.

Also effective as adhesion enhancing agents are liquid hydrophilic polymers including polyethylene glycols, nonionic polymers of ethylene oxide having the general formula:
HOCH₂(CH₂OCH₂)ₙCH₂OH
wherein n represents the average number of oxyethylene groups. Polyethylene glycols available from Dow Chemical are designated by a number such as 200, 300, 400, 600, 2000 which represents the approximate average molecular weight of the polymer, as well as nonionic block copolymer of ethylene oxide and propylene oxide of the formula:
HO(CH₄O)ₐ(C₃H₆O)_{b}(C₂H₄O)_{c}H
The block copolymer is preferably chosen (with respect to a, b and c) such that the ethylene oxide constituent comprises from about 65 to about 75% by weight, of the copolymer molecule and the copolymer has an average molecular weight of from about 2,000 to about 15,000 with the copolymer being present in the liquid tooth whitening composition in such concentration that the composition is liquid at room temperatures (23° C.).

A particularly desirable block copolymer for use in the practice of the present invention is available commercially from BASF and designated Pluraflo L1220 which has an average molecular weight of about 9,800. The hydrophilic poly(ethylene oxide) block averages about 65% by weight of the polymer.

Adhesion enhancing agents employed in compositions of various embodiments of the invention are present in an amount of from about 0 to about 20% by weight. Preferably, the adhesion enhancing agents are present in an amount of from about 2 to about 15% by weight.

### Additional Ingredients

As previously described, many other components may further be included in the whitening compositions of the present invention, and include flavors, sweetening agents, surfactants, anti-microbial agents, anti-inflammatory agents, plaque buffers, vitamins, anti-caries agents, anti-plaque agents, desensitizing agents, coloring agents, pigments and opacifying agents, for example.

In certain embodiments, nonionic surfactants are present in the whitening composition. These surfactants are preferably compatible with the whitening agents and serve as solubilizing, dispersing, emulsifying and wetting agents. In one aspect, surfactants are especially effective to solubilize a flavoring agent, if flavor is desired for the liquid whitening composition. A particularly useful nonionic surfactant is a water soluble polyoxyethylene monoester of sorbitol with a C10 to C18 fatty acid, marketed commercial under the Tween trademark. The Tween surfactants are mixtures of C10 to C18 fatty acid esters of sorbitol (and sorbitol anhydrides), consisting predominately of the monoester, condensed with about 10-30, preferably about 20, moles of ethylene oxide. The fatty acid (aliphatic hydrocarbonyl monocarboxylic acid) may be saturated or unsaturated, e.g., lauric, palmitic, stearic, oleic acids. Polysorbate 20 (e.g., Tween 20) is especially preferred and is commonly referred to as polyoxyethylene (20) sorbitan monolaurate. The nonionic surfactant constitutes about 0 to 50% by weight and preferably 0.5 to 40% by weight of the liquid composition.

In an embodiment where the whitening composition has a flavoring agent, the flavoring agent is selected from essential oils, as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Of these, the most commonly employed are the oils of peppermint, spearmint and wintergreen. The flavoring agent is preferably incorporated in the whitening composition of the present embodiment at a concentration of about 0 to about 2% by weight and more preferably about 0.1 to about 0.5% by weight.

In embodiments where the whitening composition is sweetened, a sweetening material is used as an alternative or complement to the flavoring agent. Suitable sweetening agents are water-soluble and include sodium saccharin, sodium cyclamate, xylitol, perillartien, D-tryptophan, aspartame, dihydrochalcones and the like, in concentrations of about 0.01 to about 1% by weight. Sodium saccharin is preferred.

Other ingredients which are included in various embodiments of the liquid whitening composition comprise materials commonly used in the oral care formulations. These include: antimicrobial agents, e.g., Triclosan, chlorhexidine, copper-, zinc- and stannous salts such as zinc citrate, zinc sulfate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol); anti-inflammatory agents such as ibuprofen, flurbiprofen, aspirin, indomethacine; anti-caries agents such as sodium-, calcium-, magnesium- and stannous fluoride, aminefluorides, disodium monofluorophosphate and sodium trimetaphosphate; plaque buffers such as urea, calcium lactate, calcium glycerophosphate and strontium polyacrylates; vitamins such as Vitamin C; plant extracts; desensitizing agents, e.g., potassium citrate, potassium chloride, potassium tartrate, potassium bicarbonate, potassium oxalate, potassium nitrate and strontium salts; agents effective against dental calculus such as pyrophosphate salts including the mono, di, tri and tetra alkali metal and ammonium pyrophosphate and tripolyphosphate salts; biomolecules, e.g., bacteriocins, antibodies, enzymes such as papain, glucoamylase; opacifying agents, pigments, coloring agents and fluoride ion providing salts having anti-caries efficacy such as sodium fluoride, potassium fluoride, a tin fluoride such as stannous fluoride. In another embodiment, the whitening composition comprises one or more desensitizing agents, such as potassium nitrate, citric acid, citric acid salts, strontium chloride and the like.

In one embodiment, the tooth whitening composition has the following general formula:

| Component | Content |
|---|---|
| Hydrophobic Polymer Carrier | 1 to 80 wt % |
| Adhesive Enhancing Agent | 0 to 20 wt % |
| Whitening Agent (Peroxide) | 0.5 to 50 wt % |
| Surfactant | 0 to 50 wt % |
| Flavor | 0.1 to 1 wt % |
| Other Components (can be HOH) | 0 to 10 wt % |

### Strip

The whitening composition of the present invention may be in the form of a layer of a strip prepared using a conventional solvent casting process. Strips among those useful herein comprise polymers, natural and synthetic woven materials, non-woven material, foil, paper, rubber and combinations thereof. Preferably the strip of material is substantially water insoluble. Suitable polymers include polyethylene, ethylvinylacetate, polyesters, ethylvinyl alcohol, fluoroplastics, and combinations thereof In various embodiments, the strip of material is generally less than about 1 mm (millimeter) thick, optionally less than about 0.05 mm thick, optionally about 0.001 to about 0.03 mm thick. The shape of the strip is any shape and size that covers the desired oral surface. In one embodiment, the length of the strip material is from about 2 cm (centimeter) to about 12 cm, in another embodiment from about 4 cm to about 9 cm. The width of the strip material will also depend on the oral surface area to be covered. The width of the strip is generally from about 0.5 cm to about 4 cm, in one embodiment from about 1 cm to about 2 cm. The strip material may comprise shallow pockets, optionally filled by a composition of this invention. Strips among those useful herein are disclosed in U.S. Pat. No. 6,514,484, Rajaiah et al. issued Feb. 4, 2003.

For example, in some embodiments, a strip is prepared by solvent casting, an adhesive polymer or resin such as colophonium and/or polyvinylpyrrolidone is dissolved in a sufficient amount of a compatible solvent such as ethanol. After a solution has been formed, the addition of the base whitening composition follows and any other ingredients such as sweeteners or flavors. The solution is coated onto a suitable casting carrier material from which the formed strip can be easily released from without damage. The carrier material must have a surface tension which allows the solution to spread evenly across the intended carrier width without soaking in to form a destructive bond between the two substrates. Examples of suitable carrier materials include glass, stainless steel, teflon, polyethylene impregnated kraft paper. The strip may be dried to a solid usable form in a high temperature air bath using a drying oven, drying tunnel, vacuum drier, or any other suitable drying equipment at a temperature. Thereafter the strip formed on the carrier is peeled off the carrier surface and cut into pieces of suitable size and shape for consumer use and packed into a suitable container.

To use the whitening strip for the present invention, the strip when applied to the teeth surface will adhere to the teeth in an appropriate manner and within 1 to 60 minutes, the teeth surfaces will whiten to a natural appearance as the whitening agent(s) present in the strip matrix migrate to the tooth surfaces. The composition can be applied to the tooth surface to be whitened for a plurality of minutes per day. The application of the composition can be repeated for multiple days to thereby whiten the teeth.

In this regard, the whitening strip is formed to have a width dimension suitable to cover one or more teeth in a row (upper or lower). Therefore, the whitening strip may be applied to one or more of the upper set of teeth, or to one or more of the lower set of teeth either separately or simultaneously. The length dimension of the whitening strip is determined by the amount of coverage desired. In this regard, the number of teeth which it is desired to whiten will determine the dimensions for the whitening strip. For instance, it may be desired to only whiten the front teeth, which are most easily seen by others. Accordingly, the length of whitening strip can be reduced in this case, as compared to the case where it is desired to whiten all of the teeth. The duration of application of whitening strip to the teeth will depend upon the type and concentration of the whitening agent(s), as well as the type and intensity of stain. After the teeth are whitened to the satisfaction of the user, the portions of the strip can be easily removed by rinsing the mouth with water and brushing.

### Paint-On Tooth Whitener

In one preferred embodiment, the whitening composition is applied using a “paint-on” technique. A small application device, such as a brush or spatula is coated with a composition of this invention and the composition is then placed on a tooth surface. Preferably, the composition is spread evenly on such surfaces, in sufficient quantity to deliver whitening agent(s) to the stained surfaces.

The paint-on whitening composition of the present invention is prepared in the form of a flowable viscous liquid suspension containing the whitening agent and is applied as such to the subject's teeth, by manual application, such as by painting the teeth with a soft applicator brush in the same manner as application of nail polish to a finger nail and without the intervention of a dentist or technological operations. Application by the user and evaporation or dissolution of the solvent leaves an adherent natural appearing white coating on the teeth.

In various embodiments, the layer of tooth whitening composition applied to tooth enamel contains no ingredients imparting thereto an unacceptable taste or texture, rendering it unpleasant to the user while adhering strongly to tooth enamel. The composition is sufficiently adherent to tooth enamel to remain on the teeth for the applied whitening composition enabling the applied coating to resist the forces commonly applied by the lips and tongue as well a those forces encountered during normal mastication, as upon the evaporation or dissolution of the solvent in mouth after application a hard coating of whitening composition forms in about 1 minute which coating is bonded securely to the tooth enamel to which it is applied. While the layer of applied paint-on whitening composition is in place, the user is to refrain from mastication. The whitening composition can be removed as and when required, at will, by an employment of standard oral hygiene procedures such as brushing or by rinsing with an alcoholic mouthwash.

### Methods

In one embodiment, the present invention provides a method of whitening the surface of a tooth in the oral cavity of a human or other animal subject using a tooth whitening composition comprising a whitening agent and a hydrophobic polymer carrier. The method comprises contacting the composition with the surface of the tooth. The contacting occurs for a duration of time sufficient to satisfactorily effect whitening of the teeth. Thus, the contacting occurs for a sufficient period of time to at least partially whiten teeth. This can be a period of time from about 1 minute to 2 hours or longer. In certain embodiments, the contacting is for a period of time from about 1 minute to about 5 minutes to about 45 minutes. The substantially non-aqueous tooth whitening composition is effective over a longer period of time, since it is not significantly diluted washed away in the oral cavity during the treatment time.

In another embodiment of the present invention, a method is provide for whitening a surface of a tooth in an oral cavity of a human or other animal subject which comprises preparing a liquid tooth whitening composition as previously described. The composition is applied to the tooth surface to be whitened for a plurality of minutes per day. The application is then repeated for multiple days to expose the teeth to multiple doses of whitening agent, and thus, thereby whitens the surface of the tooth.

The liquid whitening compositions of the present invention are prepared by adding and mixing the ingredients of the composition in a suitable vessel such as a stainless steel tank provided with a mixer. In the preparation of the liquid whitening composition, the ingredients are advantageously added to the mixer in the following order: liquid anhydrous silicone based pressure sensitive polymer (hydrophobic polymer carrier), peroxide whitening agent, adhesion enhancing agent and any desired flavoring or sweetener. The ingredients are then mixed to form a homogeneous dispersion/solution.

The present invention is illustrated by the following examples but is not to be limited thereby.

### EXAMPLE I

A series of liquid whitening paint-on composition are prepared using the ingredients listed in Table I below.

TABLE I

Composition

| Ingredients | A | B |
|---|---|---|
| BIO PSA (medium/high tack)* | 30.0 | 30.0 |
| Plastigel | 44.2 | 44.0 |
| Sodium percarbonate | 25 | — |
| PVP H₂O₂ cx** | — | 25.0 |
| Saccharin | 0.8 | 0.50 |
| Flavor | — | 0.50 |
| PEG 400 | — | — |
| Polyisobutene | — | — |

*Dissolved in 60% by weight dimethicone
**cx = cross-linked

The whitening compositions A and B are prepared by mixing the sodium percarbonate or PVP.H₂O₂ with the Plastigel followed by the pressure sensitive adhesive BIO PSA, saccharin, flavor, PEG 400 or polyisobutene. The liquid whitening composition of Table I can be packaged in sealed polypropylene bottles.

### EXAMPLE II

A second series of silicone polymer based pressure sensitive whitening compositions are prepared with the ingredients listed in Table II below, with the methods as described above.

TABLE II

Composition

| Ingredient | C | D |
|---|---|---|
| Ethyl acetate | 13.50 | 13.50 |
| Bio PSA (medium/high tack) | 35.00 | 35.0 |
| Pluracare L1220 | 5.00 | 5.0 |
| Sodium saccharin | 1.00 | 1.0 |
| Snow white petrolatum | 5.00 | 12.0 |
| Zinc oxide | 7.00 | — |
| Sodium percarbonate | 33.00 | 33.0 |
| Flavor | 0.50 | 0.50 |
| Total | 100.00 | 100.00 |

1. A tooth whitening composition for whitening a surface of a tooth comprising a whitening agent and a hydrophobic polymer carrier, wherein said whitening composition adheres to the tooth surface to effect whitening thereof.

2. A composition according to claim 1, wherein said hydrophobic carrier of said whitening composition is substantially non-water soluble.

3. A composition according to claim 1, wherein said whitening composition comprises from between about 1 to about 80 weight % of said hydrophobic polymer carrier.

4. A composition according to claim 1, wherein said whitening composition comprises from between about 40 to about 80 weight % of said hydrophobic polymer carrier.

5. A composition according to claim 1, wherein said hydrophobic polymer of said carrier comprises a siloxane.

6. A composition according to claim 1, wherein said hydrophobic polymer of said carrier comprises a silicone resin.

7. A composition according to claim 1, wherein said hydrophobic polymer of said carrier comprises a polyorganosiloxane.

8. A composition according to claim 1, wherein said hydrophobic polymer of said carrier is a pressure sensitive adhesive.

9. A composition according to claim 1, wherein said whitening composition comprises from between about 0.5 to about 50 weight % of said whitening agent.

10. A composition according to claim 1, wherein the whitening agent is selected from the group consisting of peroxides, chlorites and hypochlorites.

11. A composition according to claim 1, wherein the whitening agent is selected from the group consisting of polyvinylpyrrolidone/hydrogen peroxide, liquid hydrogen peroxide, urea peroxide, sodium percarbonate, sodium perborate, and mixtures thereof.

12. A composition according to claim 1, wherein said whitening composition further comprises an adhesion enhancing agent from about 0.1 to about 20 weight % of the whitening composition.

13. A composition according to claim 12, wherein said adhesion enhancing agent comprises one or more components selected from the group consisting of: waxes, nonionic polymers comprising ethylene oxide, nonionic copolymers comprising ethylene oxide and propylene oxide, polyethylene gelled mineral oil, petrolatum, hydrophobic silica containing compounds, and mixtures thereof.

14. A composition according to claim 1, wherein said whitening composition further comprises one or more of the following components: a surfactant, a flavoring agent, a desensitizing agent, an antimicrobial agent, an anti-caries agent, and an anti-calculus agent.

15 A composition according to claim 1, wherein said whitening composition comprises between about 1 to about 80 weight % of said hydrophobic carrier, between about 0.5 to about 50 weight % of said whitening agent, between about 0 to about 20 weight % of an adhesive enhancing agent; between about 0 to about 50 weight % of a non-ionic surfactant; between about 0 to about 1 weight % of a flavoring agent; and between about 0 to about 10 weight % of other components.

16. A method for whitening a surface of a tooth in an oral cavity of a human or other animal subject which comprises
(a) applying a liquid tooth whitening composition of claim 1 to the tooth surface to be whitened for a plurality of minutes per day; and
(b) repeating b for multiple days to thereby whiten the teeth.

17. A method of whitening a tooth in a mammal, the method comprising applying to the tooth a composition comprising a hydrophobic polymer carrier and a whitening agent.

18. A method according to claim 20, wherein said applying is achieved by contacting a film comprising said tooth whitening composition with a surface of a the tooth.

19. A method according to claim 20, wherein said applying is achieved by contacting a liquid form of said tooth whitening composition with a surface of the tooth.
